SEQUENCE LISTING (9 sequences)

SEQ ID NO: 1 - DNA, 1529 bp, Oryza sativa
  CDS (112)..(1023)
gccaagccctcctctcctcttcccaacactagtaggataaagccacagagagagcagtagtagtagcgagctcgccggagaacggacgatcaccggagaagggggagagagatgagcggcggtcaggacctgcagctgccgccggggttccggttccacccgacggacgaggagctggtgatgcactacctctgccgccgctgcgccggcctccccatcgccgtccccatcatcgccgagatcgacctctacaagttcgatccatggcagcttccccggatggcgctgtacggagagaaggagtggtacttcttctccccgcgagaccgcaagtacccgaacgggtcgcggccgaaccgcgccgccgggtcggggtactggaaggcgaccggcgccgacaagccggtgggctcgccgaagccggtggcgatcaagaaggccctcgtcttctacgccggcaaggcgcccaagggcgagaagaccaactggatcatgcacgagtaccgcctcgccgacgtcgaccgctccgcccgcaagaagaacagcctcaggttggatgattgggtgctgtgccggatttacaacaagaagggcgggctggagaagccgccggccgcggcggtggcggcggcggggatggtgagcagcggcggcggcgtccagaggaagccgatggtgggggtgaacgcggcggtgagctccccgccggagcagaagccggtggtggcggggccggcgttcccggacctggcggcgtactacgaccggccgtcggactcgatgccgcggctgcacgccgactcgagctgctcggagcaggtgctgtcgccggagttcgcgtgcgaggtgcagagccagcccaagatcagcgagtgggagcgcaccttcgccaccgtcgggcccatcaaccccgccgcctccatcctcgaccccgccggctccggcggcctcggcggcctcggcggcggcggcagcgaccccctcctccaggacatcctcatgtactggggcaagccattctagacgaccaaaaaaaaaaaaaaacaaccgcattggcagcaatggtgtcactgaacaccgtgcaggctagctagcttcatggccggtgaactttgactcaggcgagccgccggagttgactcaaagataattaaaagaagtgttttaagtggattggattggattagacagaggagatgaggactcgagaaaggcggcgatgagaccgtggttggggggaccctggcctggactgaacgacgacgaggcagcagcagaaagatggtgcaattgcatcgggtggcatgtcagtgtgtgtgtatagtggcatgtacatagtacatggtgattgattcggtatacagggggctagctttcctgtttctgtttcttcattggttaattattactcccattataaggtcttcttcagggttgctagcttaattaattaattaattagcccagtggttgaagtgtaagtcaaaattcatcaagtcagagactggaataatacaatacagtactgc

SEQ ID NO: 2 - protein, 303 aa, Oryza sativa
MSGGQDLQLPPGFRFHPTDEELVMHYLCRRCAGLPIAVPIIAEIDLYKFDPWQLPRMALYGEKEWYFFSPRDRKYPNGSRPNRAAGSGYWKATGADKPVGSPKPVAIKKALVFYAGKAPKGEKTNWIMHEYRLADVDRSARKKNSLRLDDWVLCRIYNKKGGLEKPPAAAVAAAGMVSSGGGVQRKPMVGVNAAVSSPPEQKPVVAGPAFPDLAAYYDRPSDSMPRLHADSSCSEQVLSPEFACEVQSQPKISEWERTFATVGPINPAASILDPAGSGGLGGLGGGGSDPLLQDILMYWGKPF

SEQ ID NO: 3 - DNA, 34 bp, Artificial
  synthetic
caggtaccgccaagccctcctctcctcttcccat

SEQ ID NO: 4 - DNA, 27 bp, Artificial
  synthetic
caggatcccctcgtcgtcgttcagtcc

SEQ ID NO: 5 - DNA, 27 bp, Artificial
  synthetic
tagaattcgacgaggagctggtgatgc

SEQ ID NO: 6 - DNA, 27 bp, Artificial
  synthetic
taggatcccctcgtcgtcgttcagtcc

SEQ ID NO: 7 - DNA, 90 bp, Artificial
  synthetic
ccccgcgcgacgtcgacaagtcgacaagtgcgaggagctagccatgtgggtcgtgcccgcgcgcgccacggcacggcaaccccggaaacg

SEQ ID NO: 8 - DNA, 24 bp, Artificial
  synthetic
ggatccctcctctcctcttcccat

SEQ ID NO: 9 - DNA, 19 bp, Artificial
  synthetic
gaattcgttcttcttgcgg

(19) United States
(12) Patent Application Publication
(10) Pub. No.: US 2010/0186108 A1
(43) Pub. Date: Jul. 22, 2010

(54) Improving Cold- and Salt-tolerant Performance of Plants with Transcription Factor Gene SNAC2 from Rice

(75) Inventors: Honghong Hu, Wuhan (CN); Lizhong Xiong, Wuhan (CN)

Correspondence Address: THOMPSON COBURN, LLP, ONE US BANK PLAZA, ST. LOUIS, MO, 63101, US

(73) Assignee: Huazhong Agricultural University

(21) Appl. No.: 12/531,001

(22) PCT Filed: Mar. 11, 2008
(86) PCT No.: PCT/CN08/00483

§ 371 Date: Feb. 19, 2010

(30) Foreign Application Priority Data
Mar. 12, 2007 (CN) 200710051654.7

(51) Int. Cl. A01H 5/00; C12N 15/82

(52) U.S. Cl. 800/278; 800/298

(57) ABSTRACT

The present invention relates to clone isolation, function confirmation and use of the SNAC2 gene from rice associated with the plant tolerance to cold and salt stress. Said gene comprises (a) a DNA sequence as shown in position 112-1023 of SEQ ID NO:1, or (b) a DNA sequence that encodes the same protein as that encoded by (a). The present invention also relates to use of said gene in increasing the tolerance of plants to drought and salt stress.

### TECHNICAL FIELD

The present invention relates to the field of plant biotechnology, and more particularly, to clone isolation, function confirmation and use of a DNA fragment (gene) from rice. Said gene is associated with plant tolerance to cold and salt stress. The cold- and salt-tolerance performance of transgenic plant is markedly improved by transferring a complete translation region (Coding sequence) of the gene linked with a strong promoter (Ubiquitin1) from corn to a plant.

### BACKGROUND ART

The growth of plants is naturally subjected to the influence of environment. For example, drought, salt injury and low temperature always lead to great reduction of crop production, thereby posing a challenge to the development of agriculture in many areas. To cope with or adapt to the adverse effect of environment, plants perceive the changes of extracellular environmental conditions and signal the cells through many pathways. In response, the cells induce expression of some responding genes to generate some functional proteins, osmoregulation substances and transcription factors for signal transmission and gene expression regulation, all of which protect the cells from stress impairment of drought, high salinity, low temperature and the like, so that plants are able to make corresponding responses to environmental changes (Xiong et al., Cell signaling during cold, drought and salt stress. Plant Cell. 14 (suppl), S165-S183, 2002). Whether those functional genes can be properly expressed during a response to an environmental stimulus is precisely regulated by regulation factors, particularly, transcription factors. It has been currently found that expression of members of transcription factor families such as AP2/EREBP, Zinc finger, Myb, bZIP, and NAC may be induced or inhibited under different environmental stresses. Therefore, they are considered to play a very important role in regulating and controlling a plant's response to environmental stress. Moreover, the isolation and identification of the transcription factors, which play a critical role in regulation and control and may be applied to genetically improve stress-resistant crops, is highly contributive to crop breeding. Presently, attempts have been made to improving plant performance during stress. For example, transgenic Arabidopsis plants overexpressing DREB1A showed increased tolerance to low temperature and drought than wild type plants (Liu Q et al., “Two transcription factors, DREB1 and DREB2, with an EREBP/AP2 DNA domains separate two cellular signal transduction pathways in drought- and low-temperature-responsive gene expression, respectively, in Arabidopsis.” Plant Cell. 1998, 10: 1391-1406). The research group of Thomashow at Michigan State University (U.S.A) also cultivated plants with enhanced cold tolerance via genetic transformation with Arabidopsis CBF1 gene.

Rice is one of the most important alimentary crops. Rice with improved cold- and salt-tolerant performance has important significance for human. Therefore, there exists an urgent need to find transcription factors associated with cold- and salt-tolerant performance so as to cultivate enhanced cold- and chilling-tolerant varieties.

### SUMMARY OF THE INVENTION

An object of the present invention is to isolate a DNA fragment containing complete encoding segments of a transcription factor gene associated with cold and salt tolerance from rice, to clone it, and to use the gene to improve the stress tolerance of rice and other plants. A structure analysis of this gene has shown that it belongs to NAC transcription factor family specific to plant, and relates to stress, as such named SNAC2.

The present invention relates to isolation and use of a DNA fragment containing SNAC2 gene, which confers plant enhanced tolerance ability in stress conditions such as low temperature and the like. Said DNA fragment is, for example, shown in SEQ ID NO: 1, the highly homologous DNA sequence substantially equivalent to SEQ ID NO: 1, or the subfragment of sequence shown in SEQ ID NO: 1 having substantially the same function.

A gene of the present invention or a homologous gene thereof can be obtained by screening a cDNA or genomic DNA library with a cloned SNAC2 gene used as a probe. The SNAC2 gene of the present invention and any DNA segments of interest or homologous DNA segments thereof may also be obtained by amplification from genomic DNA, mRNA and cDNA using PCR (polymerase chain reaction) technology. Thereby, a sequence containing SNAC2 gene may be isolated. By transforming plants with said isolated sequence in any expression vector that can direct the expression of an exogenous gene in plant, transgenic plants with enhanced tolerance to low temperature and high salinity stresses can be produced. According to the present invention, in the process of constructing the gene of the present application into the expression vector of the plant, any strong promoter or inducible promoter can be added to the position preceding the transcription initiation nucleotide, or alternatively, an enhancer may be used. Such a enhancer region can be ATG start code and start code of contiguous regions and the like, provided that the enhancer region is in the same frame as the coding sequence to ensure the translation of a complete sequence.

The expression vector bearing a SNAC2 gene of the present invention can be introduced into plant cells by conventional biotechnological techniques such as Ti plasmid, plant virus vector, direct DNA transformation, microinjection, electroporation, and the like (Weissbach, 1998, Method for Plant Molecular Biology VIII, Academy Press, New York, pp. 411-463; Geiserson and Corey, 1998, Plant Molecular Biology (2^{nd} Edition)).

The expression vector containing a SNAC2 gene of the present invention can be used to transform a host, which is a wide variety of plants including rice, to cultivate plant varieties having excellent salt- and cold-resistance.

The gene of the present invention is expressed by induction of stress, and therefore its promoter is an inducible-type promoter. By inserting both a promoter segment of the present invention and any gene of interest into an appropriate expression vector and transforming a plant host, it is feasible to induce expression of the gene under stress conditions, thereby improving the tolerance performance of the plants in response to stress.

The present invention will be further demonstrated hereunder in conjunction with specific embodiments.

### BRIEF DESCRIPTION OF THE DRAWINGS

SEQ ID No: 1 in the Sequence Listing shows the DNA fragment sequence isolated and cloned in accordance with the present invention, comprising SNAC2 gene encoding region.

FIG. 1 shows a flow chart of isolation and identification of SNAC2 gene.

FIG. 2 shows the expression level of SNAC2 gene measured by Northern hybridization at different time points under different stresses including drought, salt, cold and ABA.

FIG. 3 shows the expression of SNAC2 gene in transgenic plants, wherein with the first lane is control, and the rest are transgenically independent transgenic plants.

FIG. 4 shows growth of seedling stage SNAC2 over-expressing transgenic families in recovery phase after subjecting to low temperature stress, wherein a half of each little red pail is planted with the controls, the other half is planted with transgenic plants. Low temperature stress refers to a condition where the plant is subjected to 12 h light/12 h dark in a 4° C. incubator for 5 days, followed by growth recovery under normal conditions.

FIG. 5 shows growth of seedling stage SNAC2 over-expressing transgenic families in high salinity, wherein FIG. 5A is a picture taken from seedlings that had been germinated for 4 days before being transplanted to a MS medium containing 150 mM NaCl where it grew for 18 days, and FIG. 5B shows statistical results concerning plant height and root length (B).

FIG. 6 illustrates a trans-activation assay in yeast and a yeast single-hybrid assay that demonstrate that SNAC2 has characteristics of transcription activation and DNA binding, wherein FIG. 6A is the trans-activation assay; FIG. 6 B is the yeast single-hybrid assay.

FIG. 7 illustrates a subcellular location of SNAC2 gene in plant cells. FIG. 7A is schematic diagram of a constructed vector; FIG. 7B is visual inspections with confocal microscopy, among which FIG. 7B(i) is an observation of callus section stained with fluorescent dye propidium iodide, FIG. 7B(ii) is an image of GFP expression under green fluorescence, FIG. 7B(iii) is merged result of red and green fluorescences.

### EXAMPLES

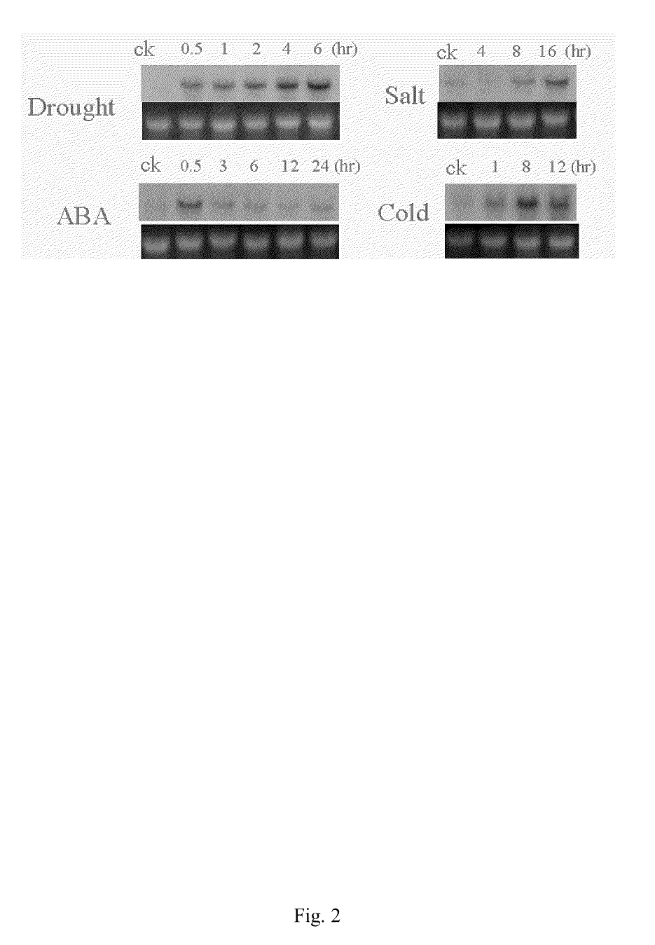

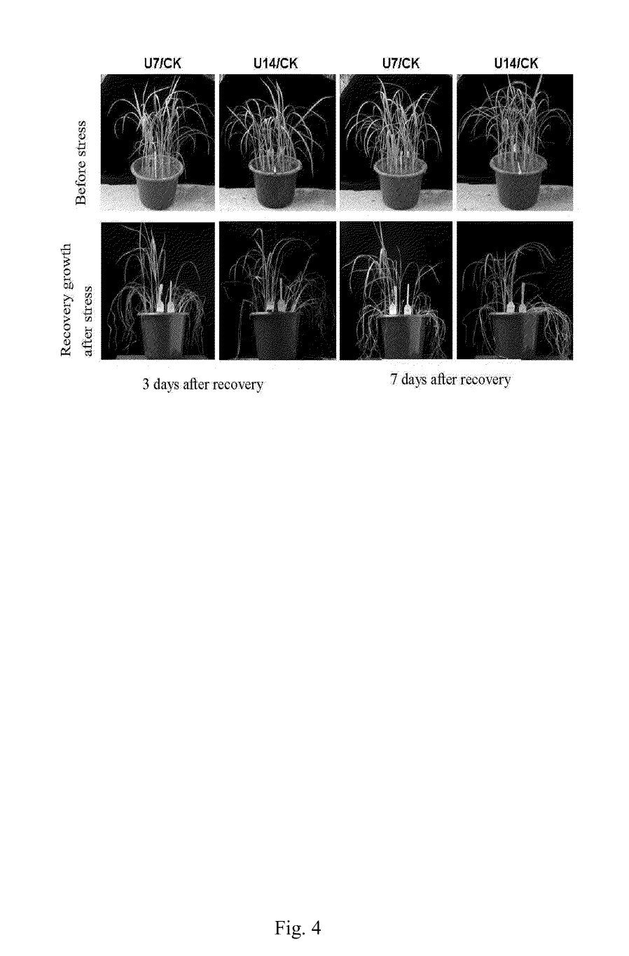

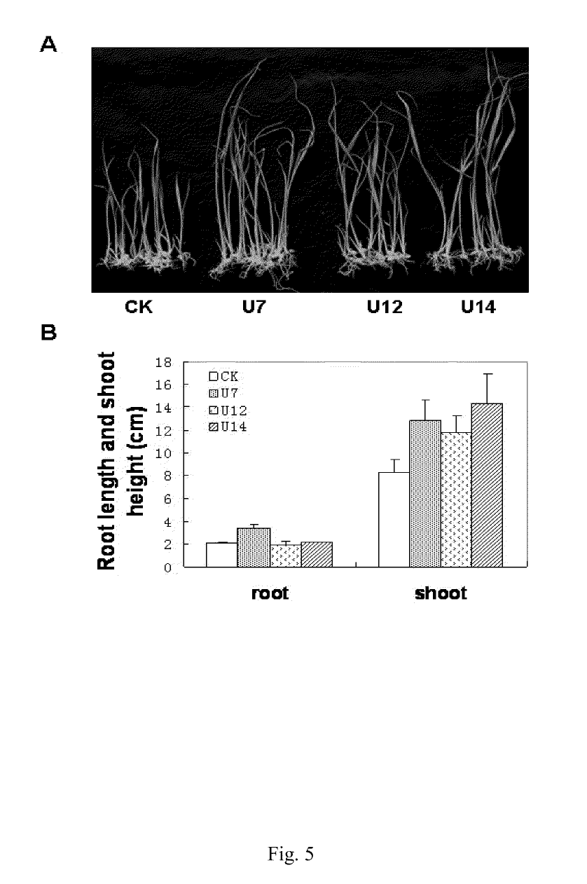

At the initial stage of the present invention the cDNA clone 99C10 derived from the rice variety MingHui 63 (a rice variety which is widely cultivated in China) was obtained. This cDNA as obtained is a full-length cDNA of SNAC2 gene, which is a transcription factor associated with drought resistance. These are the following reasons why this cDNA was used: (1) it was observed upon an analysis using a cDNA chip technique that the expression amount of cDNA clone 99C10 in the rice variety “ZhongHan 5” (a public available rice variety provided by Shanghai Academy of Agricultural Sciences, China) was increased by 3.5 times after a drought stress treatment for 15 days. The results of sequencing indicated that the gene was OsNAC6 (accession number AK068392). In view of significant difference in the expression amount of the clone after drought treatment and its functional characteristics, it was considered that the gene represented by 99C10 clone was involved in regulating and controlling expression of the gene under stresses; (2) according to the analysis of expression profile of the gene under stress (see FIG. 2), it was found that the expression level of the gene was notably increase during stress treatments; (3) the transgenic plants with over-expression of the full length gene have significantly enhanced tolerance to cold and high salinity (see FIGS. 4 and 5). All of these show that SNAC2 gene is a stress-associated gene involved in regulating and controlling the resistance not only to drought but also to high salinity and cold.

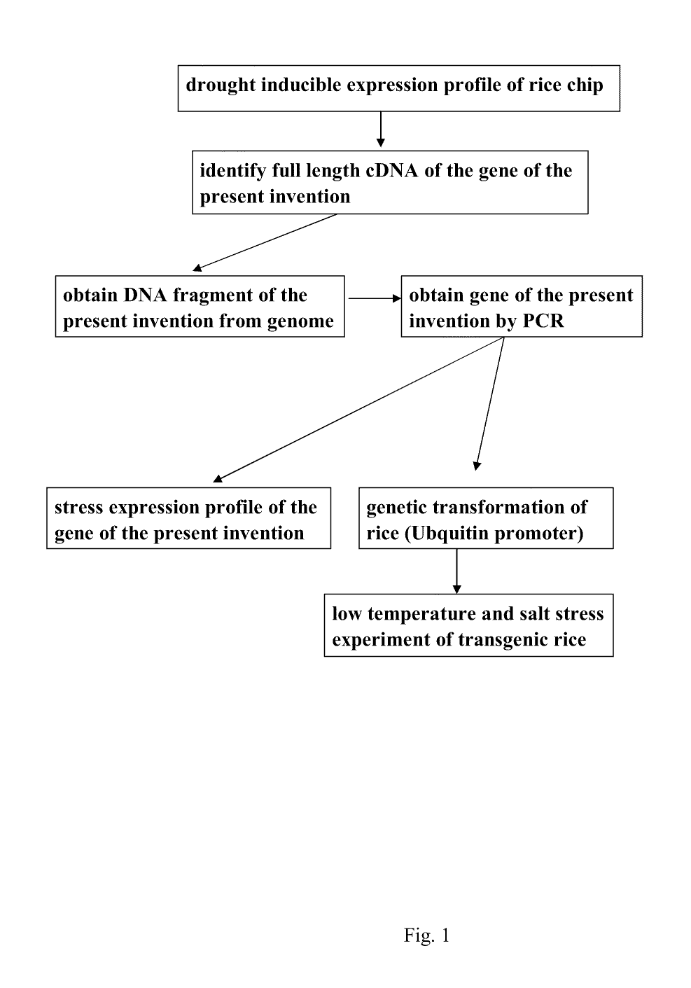

The present invention will become more apparent with the following descriptions of the examples, which are related to the methods for isolating and cloning the DNA fragment comprising the complete encoding region of SNAC2 gene and for verifying the function of SNAC2 gene based on the above results at the initial stage of the present invention (flow scheme of these methods as shown in FIG. 1). From the following description and examples, one skilled in the art can determine the basic features of the present invention, and appreciate that any changes and modifications can be furthermore made to the present invention to adapt to various uses and conditions without departing from the spirit and scope of the present invention.

### Example 1

### Isolation and Clone of DNA Fragment Containing SNAC2 Gene Segment

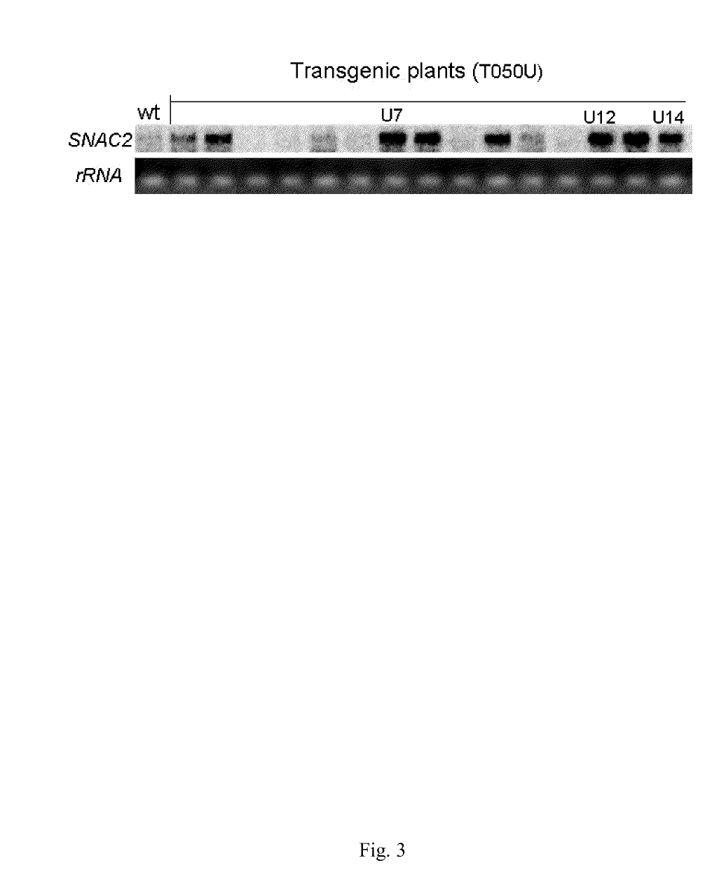

An analysis on the expression profile of drought inducible genes of the rice variety “ZhongHan 5” (a public available rice variety provided by Shanghai Academy of Agricultural Sciences, China) found a strongly drought-inducible EST (expression sequence tag), the expression amount of which increased 3.5 times or even more in the later stage of drought stress. A sequence analysis indicated that this gene was a member of the transcription factor family NAC and was a full-length sequence, corresponding to cDNA clone J013149P14 of Japan Rice full-length Database (http://cdna01.dna.affrc.go.jp). According to this cloned sequence, the primer T050F (5′-CAGGTACCGCCAAGCCCTCCTCTCCTCTTCCCAT-3′, sequence specific primer plus linker KpnI site) and T050R (5′-CAGGATCCCCTCGTCGTCGTTCAGTCC-3′, sequence specific primer plus linker BamHI) were designed, and the 1-1269 by of the clone was amplified from the variety “ZhongHan 5” by reverse transcription (FIG. 3). The amplified product is the sequence of 1-1269 by of the present invention. The acquisition of this full-length gene comprised the following steps: extracting with a TRIZOL reagent (purchased from Invitrogen Inc.) the total DNA from the leaves from the rice variety “ZhongHan 5” subjected to drought stress treatment (see the manual of above described TRIZOL reagent for the detailed extraction method); reverse transcribing with a reverse transcriptase (purchased from Invitrogen Inc.) to synthesize cDNA first chain (reacted under 65° C. for 5 min, 42° C. for 50 min, and 70° C. for 10 min); amplifying the reverse transcribed product with nested primer designed according to the sequence of cDNA clone J013149P14 (the reaction conditions were: predenaturation at 94° C. for 2 min; 30 cycles of 94° C. for 30 sec, 55° C. for 30 sec, 72° C. for 2 min; extension at 72° C. for 5 min); inserting the PCR products obtained from amplification into a pGEM-T vector (purchased from Promega Inc.); and selecting and sequencing the positive clones to obtain the desired full-length gene. Such a clone was named as PGEM-SNAC2.

### Example 2

### Detection of Inducible Expression of Rice Endogenous Gene SNAC2

The rice variety “Zhonghan 5” as a raw material was treated separately under drought, cold and high-salinity stress as well as ABA at the 3 leaf stage. The drought treatment was conducted by immersing the seedling root into 20% polyethylene glycol (trademarked as PEG6000) for 0 h, 0.5 h, 1 h, 2 h, 4 h, 6 h, before sampling. The cold treatment was conducted by placing the seedling in an incubator at 4° C. for 0 h, 1 h, 8 h, 12 h, before sampling. The high-salinity stress was conducted by immersing the seedling root in 200 mM/L NaCl solution for 0 h, 4 h, 8 h, 16 h, before sampling. The ABA treatment was conducted by immersing the seedling root in 100 μM/L ABA solution for 0 h, 0.5 h, 3 h, 6 h, 12 h, 24 h before sampling. The total RNAs of the leaves were extracted (using Trizol reagent, Invitrogen), then subjected to RNA membrane transfer (according to the experimental methods of “Molecular Cloning”, Science Press, Peking, 1999), and finally a Northern hydride with SNAC2 used as a probe. The result showed that the expression of the SNAC2 gene cloned in the present invention can be induced by drought, cold, high-salinity and ABA (shown as in FIG. 2), therefore considered a stress-associated transcription factor.

### Example 3

### Construction and Transformation of SNAC2 Gene Over-Expression Vector

The above Example 2 showed that the expression of the SNAC2 gene of the present invention can be induced by drought, cold, high-salinity and ABA. In order to better illustrate the function of this gene, the SNAC2 gene was over-expressed in rice and verified by the phenotype of transgenic plants. The process comprises the following steps of: double digesting the positive clone pGEM-SNAC2 plasmid obtained in Example 1 with BamHI and KpnI; recovering exogenous fragments; at the same time, enzymatically cleaving the genetic transformation vector pU1301 with the corn strong promoter Ubiquitin1 (which is reconstructed based on a common vegetable genetic transformation vector pCAMBIA1301 from Australia CAMBIA Laboratory (Center for the Application of Molecular Biology to International Agriculture), carrying corn strong promoter Ubiquitin1 with constitutive and over-expression characteristics, mediated by Agrobacterium) by the same way; exacting and purifying the enzymatically cleaved products with chloroform:isopentanol (24:1) after enzymatically cleavage. The linkage reaction was conducted by using the enzymatic cleavage fragments comprising SNAC2 gene and the enzymatically cleaved pU1301 vector (shown as the figure below), transforming E. coli DH10β (purchased from Invitrogen Inc.). The positive clones were screened out by enzymatically cleavage to obtain a transformed vector.

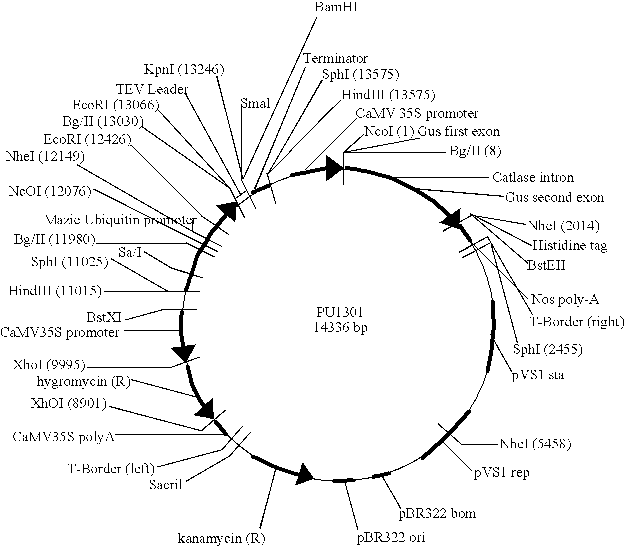

The transformed vector was introduced into the rice variety “ZhongHua 11” (a publicly available rice variety provided by China National Rice Research Institute) using a rice genetic transformation system mediated by Agrobacterium. A transgenic plant was then finally obtained by precultivation, infestation, co-cultivation, screening the callus with hygromycin resistance, differentiation, rooting, seedling establishment and transplanting. The rice (japonica rice subspecies) genetic transformation system mediated by Agrobacterium was optimized on the basis of the method reported by Hiei, et al. (See: Efficient transformation of rice, Oryza sativa L., mediated by Agrobacterium and sequence analysis of the boundaries of the T-DNA, 1994, Plant Journal 6:271-282).

The obtained transgenic rice plant was designated as T050U. The present invention obtained in total 23 independent transgenic rice plants.

### Example 4

### Cold Resistance Screening of the SNAC2 Gene Transgenic

### T2 Family in Seedling Stage

In order to verify whether the cold resistance of transgenic rice plants was enhanced and whether such a enhancement was related to an introduced SNAC2 gene, the expression of SNAC2 gene in a portion of transgenic rice plants was detected by Northern hybridization technology in the present invention (FIG. 3 shows the Northern hybridization results, wherein the method was the same as used in Example 2), and a portion of families of T2 generation plants of the present invention was screened for the cold resistance. The specific steps were as follows: the seeds of T2 generation families were germinated in MS medium containing 50 mg/ml hygromycin for 5 days, after which seedlings having substantially the same level of germination were transplanted into little red pails, one half of which was planted with transgenic over-expressed plants, and the another half was planted with wild-type control plants. When the plants grew to 4-leaf stage, they were subjected to low temperature treatment at 4° C. After 5 days of treatment, it was observed that no significant changes occurred to the phenotypes of either the transgenic plants or to the control plants. However, after they were shifted to normal conditions to recover growth for 3 days, it was observed that most of the control plants had leaves that were curled and became wilted, while only a few of leaves of the transgenic plants got wilted; at 7 days after the growth recovery, almost all of the control plants died, while, in contrast, almost 50% of the transgenic over-expressed family survived (FIG. 4). This proved that SNAC2 gene was without doubt related to cold resistance, and its over-expression could enhance the cold resistance of transgenic plants, and that the enhancement of resistance of transgenic rice plants was surely related to the introduced SNAC2 gene.

### Example 5

### Salinity Resistance Screening of SNAC2 Gene Transgenic T2 Family in Seedling Stage

It was proved in Example 4 that the cold resistant performance of SNAC2 gene transgenic plants of the present invention in seedling stage was significantly higher than that of the control. In order to verify whether SNAC2 is capable of protecting plants against other stresses, growth performance of plants in high-salinity environment was compared in this example. The comparison is taught as follows: T2 generation of transgenic over-expression family was germinated in MS medium containing 50 mg/l hygromycin for 4 days, then the transgenic seedlings and the control seedlings, which had the same growth, were transplanted to little square boxes having MS medium containing 150 mmol/L NaCl to keep growing. Growth was observed, and 18 days later, a measure was made to the root length and plant height of each young seedling. It was found that there was no difference in root length between the SNAC2 over-expression or SNAC2 inducible expression transgenic plant and the control plant grown in the high salinity condition. However, there was an apparent difference in the plant height. The growth of the control plant was substantially inhibited in the high salinity condition, reaching only 60% of that of the transgenic over-expression family (FIG. 5). This showed that the SNAC2 over-expression transgenic seedling had higher salinity tolerance than the control plant, suggesting that the SNAC2 transgenic plant of the present invention can substantially improve salinity resistance in the plants.

### Example 6

### SNAC2 Gene Having Transcription Activating and DNA Binding Properties

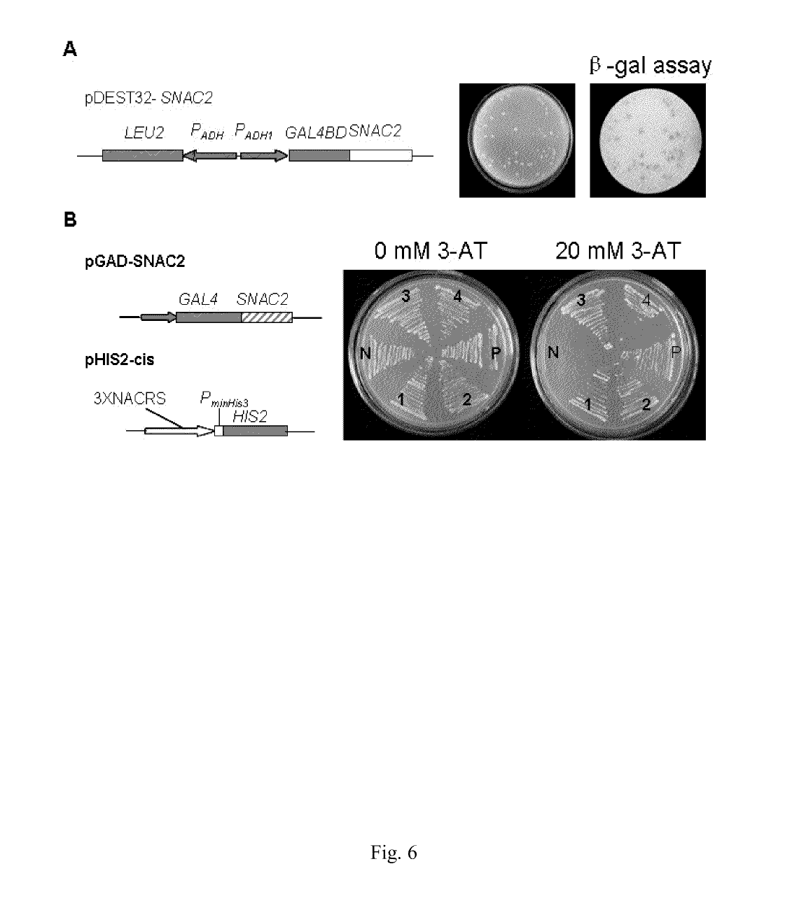

Transcription factors have transcription activating and DNA binding properties. Specifically, transcription factors bind cis acting elements of downstream gene promoters in case of signaling or stress inducement, and thereby initiate the expression of downstream target genes. Since the gene of the present invention is an inducible transcription factor, in order to verify whether the SNAC2 gene of the present invention has transcription activating and DNA binding properties, a trans-activation assay and a yeast one hybrid assay were conducted in the present example to verify the DNA binding activity and transcription regulating (activating) function of a SNAC2 protein as a transcription factor. At first, the SNAC2 gene was constructed into a yeast GAL4-DB fusion expression vector pDEST32 (purchased from Invitrogen Inc.), which was used to transform a yeast cell Y187 (purchased from CLONTECH Inc.). Then, a β-Galactosidase activity assay was conducted to determine the expression of reporter gene LacZ based on whether the yeast colony turned blue, thereby determining whether the gene had activation function. The assay results showed that the gene of the present application does activate transcription (FIG. 6A). The research results of Hu et al. (“Overexpressing a NAM, ATAF, and CUC (NAC) transcription factor enhances drought resistance and salt tolerance in rice.” Proc Natl Acad Sci USA, 2006, 103: 12987-12992) suggest that SNAC1 could bind to a DNA sequence similar to a NACRS (NAC recognition site) identified in Arabidopsis. In order to further verify whether the other NAC type protein SNAC2 in rice could also bind to this sequence, the applicants further examined in the present example the interaction between the SNAC2 protein and the DNA containing sequence of CATGTG and CACG in OsERD1 promoter in yeast. The yeast cell Y187 was co-transformed with pHIS-cis and expression vector pGAD-SNAC2 in which the full-length SNAC2 encoding sequence was fused to GAL4-activation domain of yeast vector pGAD-RecT7 by the present applicants. At the same time the positive control (pHIS53/p53GAD) and negative control (pGAD-SNAC2/pHIS53) were transformed. The result showed that the desired transformer, negative control and positive control can grow in petri dishes SD/Leu⁻/Trp⁻/His⁻ for yeast without 3-AT. However, when 20 mmol/L 3-AT was added, the negative control transformer could not grow in SD/Leu⁻/Trp⁻/His⁻ medium, while the positive control and the desired transformer can grow well (FIG. 6B). This result showed that SNAC2 can also recognize and bind to the sequence of OsERD1 promoter region containing CATGTG and CACG, and had transcription activating function in yeast cell. The result also suggested that the NAC protein of rice can recognize sequences similar with that recognized by Arabidopsis NAC protein. All of these experiments showed that the SNAC2 gene has transcription activating and DNA binding properties.

The specific steps for executing the trans-activation assay were as follows:

1. The full-length SNAC2 gene was fused to the yeast expression vector pDEST32 (purchased from Invitrogen Inc.).

The gene primers were designed according to the open reading frame of pDEST32 vector based on the full-length cDNA clone sequence (using software Primer 5.0). The obtained PCR products were purified by PEG8000, before subjected to a BP recombination reaction with an intermediate vector pDONR221 (purchased from Invitrogen Inc.). The reaction system was in a volume of 5 μl, including 200 ng of PCR product, 50 ng of pDONR221, 2 μl of 5×BP Clonase Reaction Buffer and 2 μl of BP Clonase Mix, and was incubated for reaction at 25° C. for about 5 h. An E. coli DH10β (purchased from Invitrogen Inc.) was transformed with reaction product to screen out the positive clones. The desired positive clone plasmids were finally subjected to a LR recombination reaction, such that the gene fragment carried by the desired positive clone plasmid was fused to yeast expression vector pDEST32. Said LR recombination reaction is taught as follows: an E. coli DH10β (purchased from Invitrogen Inc.) was transformed at 25° C. for about 5 h with 100 ng of positive plasmid of BP reaction, 50 ng of pDEST32, 2 μl of 5×LR Clonase Buffer and 2 μl of LR Clonase Mix, and the positive clones were screened.

2. Preparation and transformation of competent yeast (CLONTECH, Yeast Protocols Handbook) by lithium acetate (LiAc) method

1) Reagent and Formula

A. YPD medium:

| | |
|---|---|
| 20 g | Difco peptone |
| 10 g | Yeast extract |
| 20 g | glucose |

diluted with distilled water to 1 L, and sterilized for 15 minutes.

B. SD/Leu medium:

| | |
|---|---|
| 6.7 g | Yeast nitrogen base without amino acids |
| 20 g | Agar powder |
| 20 g | glucose |
| 0.69 g | -Leu DO Supplement (purchased from CLONTECH Inc.) |

diluted with distilled water to 1 L, and sterilized for 15 minutes.

C. 10 TE buffer: 0.1 M Tris-HCl, 10 mM EDTA, pH 7.5, sterilized

D. 10 LiAc: 1M lithium acetate, pH 7.5, sterilized

E. PEG/LiAc solution

| | Final Conc. | To prepare 10 ml of solution |
|---|---|---|
| PEG4000 | 40% | 8 ml of 50% PEG |
| TE buffer | 1X | 1 ml of 10X TE |
| LiAc | 1X | 1 ml of 10X LiAc |

2) Steps:

A. Single yeast colony with diameter of 2-3 mm was scattered with 1 ml YPD solution, and then transferred to a triangular flask containing 10 ml YPD medium.

B. Cultured under the rotation of 250 rpm at 30° C. for 16-18 h, such that OD600>1.5.

C. About 5 ml of the above-mentioned yeast solution was transferred to another triangular flask containing 50 ml YPD medium, and detected concentration to get OD600=0.2-0.3.

D. Cultured at 30° C. for 3 h (230 rpm), at this point OD600=0.4-0.6 (if OD600<0.4, the culture maybe get in trouble).

E. The yeast solution was transferred into a 50 ml centrifuge tube, and centrifuged at 1000×g for 5 minutes at room temperature.

F. The supernatant was discarded, the cells were resuspended with sterilized double distilled water, and centrifuged at 1000×g for 5 minutes at room temperature.

G. The supernatant was discarded, the yeast cells were mixed homogenously with 1 ml fresh prepared 1×TE/1×LiAc.

H. 200 ng fusion plasmid DNA was placed into a 1.5 ml centrifuge tube, and 100 μl of yeast competent cells were added and mixed homogeneously, then 600 μl PEG/LiAc was added, mixed homogeneously by centrifugation at high speed, and cultured at 30° C. for 30 min (200 rpm).

I. 70 μl DMSO (100%) was added, mildly reversed for several times, placed in 42° C. water bath for 15 min, and then placed on ice for 2 min.

J. Centrifuged at 14000 rpm for 5 seconds at room temperature, the supernatant was discarded, and the cells were scattered with 500 μl 1×TE buffer.

K. 100 μl transformed cells were uniformly coated on -Leu/SD plate, inversion cultured in 30° C. incubator for 2-4 days, until clones appeared.

3. Verification of transcription activity of SNAC2 gene and deletion mutant thereof based on the expression of reporter gene LacZ in beta-galactosidase assay

1) Reagent and Formulation

A. Z buffer

| | |
|---|---|
| Na₂HPO₄•7H₂O | 16.1 g/L |
| NaH₂PO₄•H₂O | 5.5 g/L |
| KCl | 0.75 g/L |
| MgSO₄•7H₂O | 0.246 g/L |

adjusted pH to 7.0, and sterilized.

B. X-gal stock solution (20 mg/ml)

C. Z buffer solution/X-gal solution:

| | |
|---|---|
| 100 ml | Z buffer: |
| 0.27 ml | β-mercaptoethanol |
| 1.67 ml | X-gal stock solution |

2) Steps:

A. The transformed clone was allowed to grow to 1-3 mm (30° C., 2-4 days)

B. Round sterilized Watman filter paper of appropriate size was placed on 10 cm asepsis plate, about 2.5-5 ml Z buffer/X-gal solution was added to wet the filter paper, and bubble was avoided.

C. Another clean, sterilized filter paper was placed by forceps on the petri dish with growing clone, and the filter paper was slightly pressed in order to adhere the clone to the filter paper.

D. When the filter paper was wetted, it was uncovered by forceps, and the filter paper was placed into liquid nitrogen for 10 sec with the surface adhered with the clones facing up, then it was thawed at room temperature in order to break the yeast cells.

F. The filter paper with surface adhered with clones facing up was carefully placed onto the previously wetted filter paper, and bubble was avoided.

G. The filter paper was placed at 30° C. (30 min-8 hr), and whether the gene had the activation function was determined according to the occurrence of blue spot.

The specific implementing steps of yeast one hybrid assay were as follows:

1. The full length SNAC2 gene was fused to the yeast expression vector pGAD-Rec2 (purchased from CLONTECH Inc.).

The gene primers (5-TAGAATTCGACGAGGAGCTGGTGATGC-3, specific primer plus EcoRI enzymatically cleavage site) and (5-TAGGATCCCCTCGTCGTCGTTCAGTCC-3, specific primer plus BamHI enzymatically cleavage site) were designed according to the open reading frame of pGAD-Rec2 vector based on the full length cDNA clone sequence. The obtained PCR products were double digested, purified by chloroform: isopentanol and linked to the vector pGAD-Rec2 subjected to same double digestion. E. coli DH10β (purchased from Invitrogen Inc.) was transformed, and the positive clones were screened and verified with same enzyme digestion method, yielding the yeast transformation vector pGAD-SNAC2.

2. The 3 repeats of 90 by OsERD1 promoter region sequence containing the core sequence CATGTG and CACG was linked into vector pHIS2 (purchased from CLONTECH Inc.).

The 3 tandem repeats of 90 by OsERD1 promoter region (5′-CCCCGCGCGACGTCGACAAGTCGACAAGTGCGAGGAGCTAG CCATGTGGGTCGTGCCCGCGCGCGCCACGGCACGGCAACCCCG GAAACG-3′) comprising the core sequence CATGTG and CACG was synthesized inhouse with EcoRI and Sad site at both end, and it was directedly linked into yeast vector pHIS2. The positive clones were verified and screened with same enzyme digestion method, yielding the yeast transform vector pHIS2-cis.

3. The competent cells were prepared and the transformation was accomplished with a yeast vector (the method was same as trans-activation assay). The cells transformed with desired binary vector (at the same time preparing positive control and negative control) were coated on petri dish of SD/Leu-/Trp-, then cultured in incubator at 30° C., until size of colony was about 2 mm.

4. The same colony was streak cultured in a petri dish of SD/Leu-/Trp-/His-containing 0 mM, 10 mM, 20 mM, 30 mM and 40 mM 3-AT, and growth performance of colony was observed.

### Example 7

### Subcellular Localization of SNAC2

In order to determine the expression location of the SNAC2 gene in a cell, a GFP-NLS (nuclear location signal) fusion protein was constructed. That is, the gene expression profile in cell was determined according to the expression of GFP. It was known that the nuclear location signal (NLS) of the NAC gene may locate at 71-83 AA according to the previously published articles on NAC gene (Mild Fujita, Kazuo Shinozaki et al., A Dehydration-induced NAC protein, RD26, is involved in a novel ABA-dependent stress-signaling pathway.” Plant J (2004) 39, 863-876, and Honghong Hu et al., “Over expressing a NAM, ATAF, and CUC (NAC) transcription factor enhances drought resistance and salt tolerance in rice.” Proc Natl Acad Sci USA, 2006, 103: 12987-12992). In this context, the subcellular location of the gene was determined according to the expression region in cell of this sequence fused with GFP. The 1-144 AA fragment of the sequence of the present invention was fused to a pCAMBIA1391-GFP vector (bearing ubiquitin1 promoter), whereby the cellular location of SNAC2 protein can be deduced according to the expression location of GFP in cell in P_{SNAC2}:Δ SNAC2-GFP transgenic plants. The pCAMBIA1391-EGFP vector (see figure below) was reconstructed based on the pCAMBIA1391 (a plant genetic transformation vector commonly used in the world), wherein the carried GUS gene was replaced with EGFP gene, with Ubiquitin1 promoter preceding GFP. The pCAMBIA1391 vector was from Australia CAMBIA Laboratory (Center for the Application of Molecular Biology to International Agriculture) and is public available.

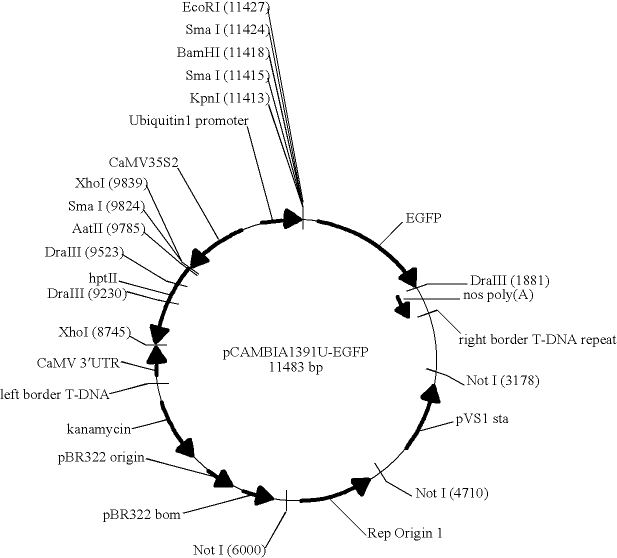

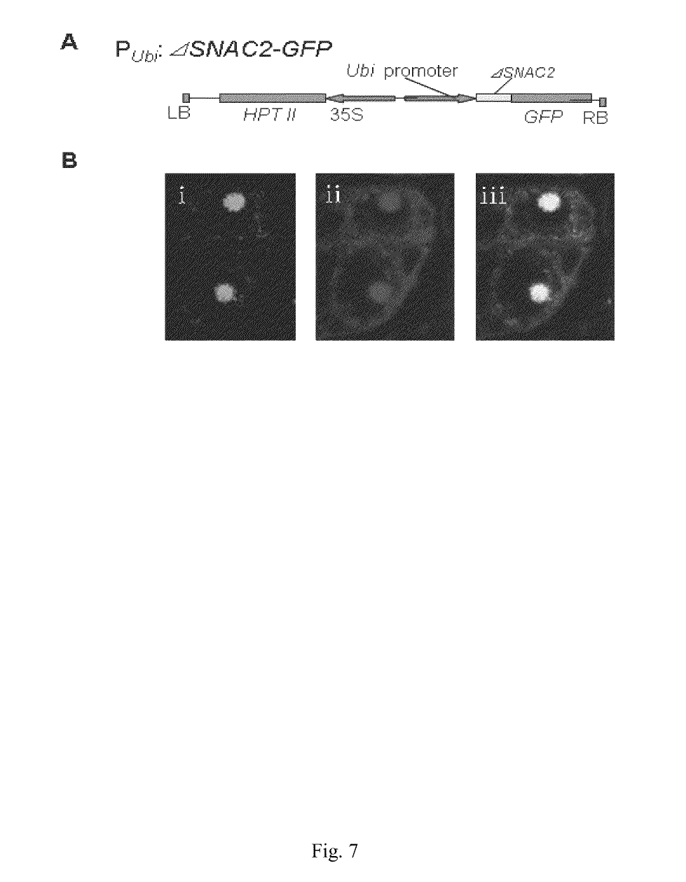

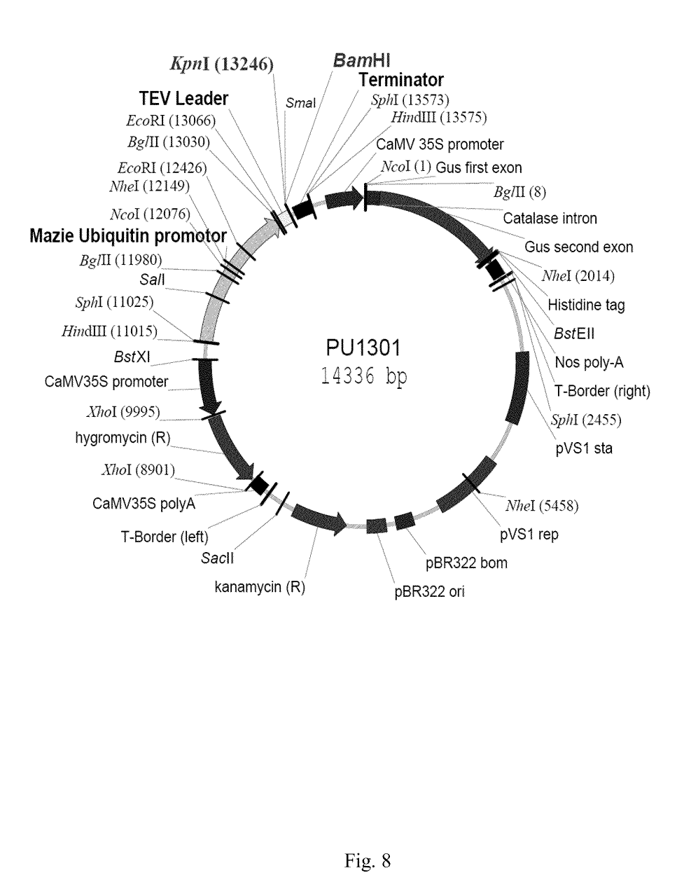

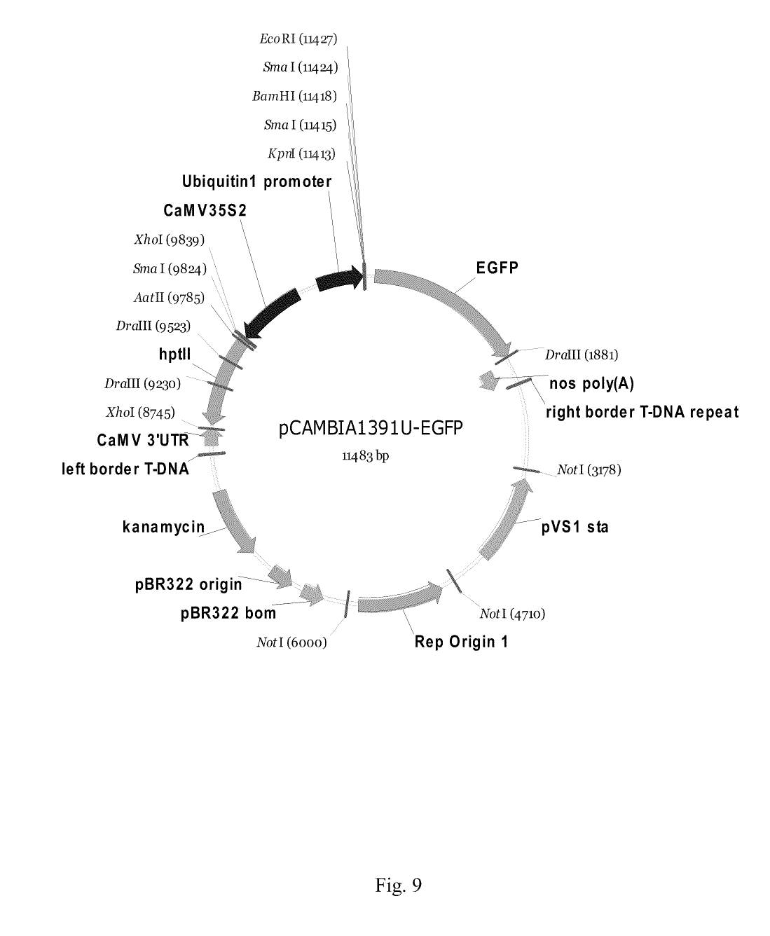

The method for the construction of the fused gene vector was described as follows: the primers PF (5-GGATCCCTCCTCTCCTCTTCCCAT, plus linker BamHI site) and PR (5-GAATTCGTTCTTCTTGCGG, plus linker EcoRI) were designed; the vector pGEM-SNAC2 constructed in above Example 1 was used as template. The SNAC2 gene was amplified by means of an amplification program of predenaturation at 94° C. for 3 min; 30 cycles of 94° C. for 30 sec, 55° C. for 30 sec, 72° C. for 3 min; extension at 72° C. for 5 min; and the amplified product was double digested by EcoRI and HindIII and linked into a pCAMBIA1391-EGFP vector that had been subjected to the same double digestion. ° The rice callus was transformed with the fusion vector p1391-GFP-NLS using an agrobacterium mediated genetic transformation method (same as the method used in Example 3), the callus with resistance was obtained under hygromycin selection pressure (specific methods as described in Example 3), and the expression of GFP was observed under fluorescence microscope (see FIG. 7A). The expressed resistance callus was sectioned and observed under confocal microscope to determine the intracellular expression of GFP. FIG. 7B shows that GFP is expressed only in nuclei under the observation of confocal microscope, which indicates that the sequence of 1-144AA already includes the NLS, so GFP could be localized in the nuclei, i.e., the SNAC2 protein was localized in nuclei. This example proved that the 1-144AA fragment of the sequence according to the present invention includes an intact NLS, and the SNAC2 protein localizes in cell nucleus.

1. A transformed plant comprising a recombinant DNA construct comprising a promoter functional in a plant cell positioned to provide for expression of a polynucleotide having a sequence with at least about 70%, 75%, 80%, 85%, 90%, or 95% identity to those in positions 112-1023 of SEQ ID NO:1.

2. A transformed plant comprising a recombinant DNA construct comprising a promoter functional in a plant cell positioned to provide for expression of a polynucleotide encoding a polypeptide at least about 70%, 75%, 80%, 85%, 90%, or 95% identical to that encoded by the polynucleotide sequence in positions 112-1023 of SEQ ID NO:1.

3. The transformed plant according to claim 1, wherein said plant is a crop plant.

4. A method of producing a transformed plant having an improved property, wherein said method comprises transforming a plant with a recombinant construct comprising a promoter functional in a plant cell positioned to provide for expression of a polynucleotide encoding a polypeptide useful for improving plant cold tolerance, drought tolerance, or salt tolerance, wherein said polynucleotide has a sequence with at least about 70%, 75%, 80%, 85%, 90%, or 95% identity to those in positions 112-1023 of SEQ ID NO:1.

5. A method of producing a transformed plant having an improved property, wherein said method comprises transforming a plant with a recombinant construct comprising a promoter functional in a plant cell positioned to provide for expression of a polynucleotide encoding a polypeptide useful for improving plant cold tolerance, drought tolerance, or salt tolerance, wherein said polypeptide is at least about 70%, 75%, 80%, 85%, 90%, or 95% identical to that encoded by the polynucleotide sequence in positions 112-1023 of SEQ ID NO:1.

6. The method according to claim 4, wherein said transformed plant is a crop plant.

7. A plant exhibiting an improved property as compared to the control plant, wherein the altered trait is selected from the group consisting of greater cold tolerance, greater tolerance to water deprivation, and greater salt tolerance, or combinations thereof, wherein the plant has greater expression or activity of a polypeptide encoded by a polynucleotide that has at least about 70%, 75%, 80%, 85%, 90%, or 95% identity to those in positions 112-1023 of SEQ ID NO:1.

8. A plant exhibiting an improved property as compared to the control plant, wherein the altered trait is selected from the group consisting of greater cold tolerance, greater tolerance to water deprivation, and greater salt tolerance, or combinations thereof, wherein the plant has greater expression or activity of a polypeptide at least about 70%, 75%, 80%, 85%, 90%, or 95% identical to that encoded by the polynucleotide sequence in positions 112-1023 of SEQ ID NO:1.

9. The transformed plant according to claim 7, wherein said plant is a crop plant.

10. The transformed plant according to claim 1, wherein said plant has enhanced salt resistance.

11. The transformed plant according to claim 1, wherein said plant has enhanced cold resistance.

12. The transformed plant according to claim 1, wherein said plant has enhanced salt resistance and enhanced cold resistance.

13. The transformed plant according to claim 1, wherein said polynucleotide comprises a sequence of positions 112-1023 of SEQ ID NO:1.

14. The transformed plant according to claim 2, wherein said plant has enhanced salt resistance.

15. The transformed plant according to claim 2, wherein said plant has enhanced cold resistance.

16. The transformed plant according to claim 2, wherein said plant has enhanced salt resistance and enhanced cold resistance.

17. The transformed plant according to claim 2, wherein said polynucleotide encodes a polypeptide identical to that encoded by the polynucleotide sequence in positions 112-1023 of SEQ ID NO:1.

18. The transformed plant according to claim 2, wherein said plant is a crop plant.

19. The method according to claim 5, wherein said transformed plant is a crop plant.

20. The transformed plant according to claim 8, wherein said plant is a crop plant.
